(19) United States
(12) Patent Application Publication
(10) Pub. No.: US 2024/0358929 A1
(43) Pub. Date: Oct. 31, 2024

(54) Plunger Rod

(71) Applicant: SHL Medical AG, Zug (CH)

(72) Inventor: Chun Chang, Nacka Strand (SE)

(21) Appl. No.: 18/687,952

(22) PCT Filed: Aug. 17, 2022
(86) PCT No.: PCT/EP2022/072885

§ 371 Date: Feb. 29, 2024

(30) Foreign Application Priority Data
Aug. 31, 2021 (EP) 21193932.7

(51) Int. Cl. A61M 5/315

(52) U.S. Cl. CPC A61M 5/31511

(57) ABSTRACT

The present disclosure provides a plunger rod for a medicament delivery device. The plunger rod includes a base at the proximal end of the plunger rod, a first arm extending from the base, and a second arm extending from the base. Each of the first arm and the second arm is flexible and configured to transform from a first rolled-up configuration to a second extended configuration.

### CROSS-REFERENCE TO RELATED APPLICATIONS

The present application is a U.S. National Phase Application pursuant to 35 U.S.C. § 371 of International Application No. PCT/EP2022/072885 filed Aug. 17, 2022, which claims priority to European Patent Application No. 21193932.7 filed Aug. 31, 2021. The entire disclosure contents of these applications are herewith incorporated by reference into the present application.

### TECHNICAL FIELD

The present disclosure relates to a plunger rod for medicament delivery devices. In particular, the present disclosure relates to a plunger rod that transforms from a rolled-up configuration to an extended configuration.

### BACKGROUND

Medicament delivery devices often comprise syringes or cartridges for storing a medicament. Usually the medicament delivery devices comprise an elongate single piece plunger rod whereby the length of the single piece plunger rod must be at least long enough to push a stopper the full length of the barrel. Usually the syringes are prefilled with a fluid medicament and act as the storage container for the fluid medicament, therefore the medicament delivery device has to accommodate the prefilled syringe barrel and its full-length single piece plunger rod.

Medicament delivery devices such as auto-injectors, safety syringes and pen-injectors are generally known for the self-administration of a medicament by patients without formal medical training. As just one example, patients suffering from diabete7s may require repeated injections of insulin. Other patients may require regular injections of other types of medicaments, such as a growth hormone or biological medicaments. Therefore, there is a demand to design medicament delivery devices with multiple automatic functions and predetermined dosage for ease of patient use. While there are a variety of delivery mechanisms and syringe designs available, alternative plunger rod designs are desirable for various applications and to reduce costs associated with manufacturing and packaging of medical devices.

The patent document WO2012056265A1 relates to a piston rod for an injection device, comprising first and second legs, each leg being foldable on itself and comprising a proximal region, said piston rod being in one of a folded position and an unfolded position, said piston rod comprising coupling means for coupling the proximal region of the first leg to the proximal region of the second leg when said piston rod is in said unfolded position. The present disclosure also relates to a kit comprising said piston rod.

U.S. Pat. No. 8,172,813 B2 discloses medical devices comprising a syringe barrel, a stopper and a two-piece plunger rod. According to one or more embodiments, the two-piece plunger rod includes a first plunger rod piece slidably mounted to a second plunger rod piece. The first and second plunger rod pieces include locking elements which allow the length of the plunger rod to be extended during use or injection or compressed during storage or packaging. In one or more embodiments, the medical device is further packaged in a compressed position.

### BRIEF DESCRIPTION

A first aspect concerns a plunger rod for a medicament delivery device, wherein the plunger rod comprises a base at the proximal end of the plunger rod, a first arm extending from the base, a second arm extending from the base, wherein each of the first arm and the second arm is flexible and is configured to transform from a first rolled-up configuration to a second extended configuration.

Optionally, each of the arms comprises an engagement mechanism.

Optionally, the engagement mechanism comprises two or more first protrusions on the first arm and two or more second protrusions on the second arm, wherein each of the two or more first protrusions is spaced apart from the other first protrusions, and wherein each of the two or more second protrusions is spaced apart from the other second protrusions.

Optionally, the two or more first protrusions are offset from the two or more second protrusions so that the two or more first protrusions fit between the two or more second protrusions when the first arm and the second arm are in the second extended configuration.

A second aspect concerns a plunger rod assembly comprising a plunger rod as described above wherein the plunger rod assembly further comprises a first support wheel configured to roll up the first arm and a second support wheel configured to roll up the second arm.

Optionally, the first support wheel comprises a first holding mechanism for holding the distal end of the first arm, wherein the holding mechanism is configured to hold the first arm when the first arm is rolled up on the first support wheel, and wherein the second support wheel comprises a second holding mechanism for holding the distal end of the second arm, and the second holding mechanism is configured to hold the second arm when the second arm is rolled up on the second support wheel.

Optionally, the first support wheel is a gear wheel comprising circumferentially outwardly protruding first teeth spaced out around the outer circumference of the first support wheel. Optionally, the second support wheel is a gear wheel comprising circumferentially outwardly protruding second teeth spaced out around the outer circumference of the second support wheel.

Optionally, the plunger rod assembly further comprises an actuation gear wheel wherein the actuation gear wheel comprises circumferentially outwardly protruding actuation teeth on the outer circumference of the actuation gear wheel and is configured to engage either the first support wheel or the second support wheel.

Optionally, the actuation gear wheel is configured to turn the first or the second support wheel when actuated, thereby unwinding the second arm and moving the base, which results in the first arm also unwinding, thereby transforming both the first arm and the second arm from a first rolled-up configuration to a second extended configuration, wherein in the second configuration, the first arm and the second arm form together an extended rigid plunger rod.

Optionally, the base is configured to exert force and displace a plunger within a barrel of a medicament container when the first arm and the second arm move from the first coiled up configuration to the second extended configuration, thereby expelling a medical fluid.

A third aspect concerns a medicament delivery device comprising a plunger rod as described above or a plunger rod assembly as described above.

Optionally, the medicament delivery device comprises a medicament container; a plunger in the medicament container; a housing; and a medicament delivery member.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

These and other aspects of and advantages with the present invention will become apparent from the following detailed description and from the accompanying drawings.

### BRIEF DESCRIPTION OF THE DRAWINGS

Embodiments of the present disclosure will now be described by way of example only and with reference to the accompanying drawings, in which:

FIG. 1 shows a perspective of the plunger rod and partially a syringe;

FIG. 2 shows a side view of the plunger rod introduced into the syringe barrel; and

FIG. 3 shows a side view of the plunger rod of FIG. 2 in a final state.

### DETAILED DESCRIPTION

One aspect of this disclosure concerns a plunger rod for a medicament delivery device, wherein the plunger rod comprises a base at the proximal end of the plunger rod, a first arm extending from the base, a second arm extending from the base, and wherein each of the first arm and the second arm is flexible and configured to transform from a first rolled-up configuration to a second extended configuration.

A medicament delivery device may comprise a housing, the housing being configured to receive a medicament container, and a medicament delivery member connected to the medicament container. The medicament delivery device comprises the plunger rod and a plunger rod assembly for actuating the plunger rod to transform from a first rolled-up configuration to a second extended configuration.

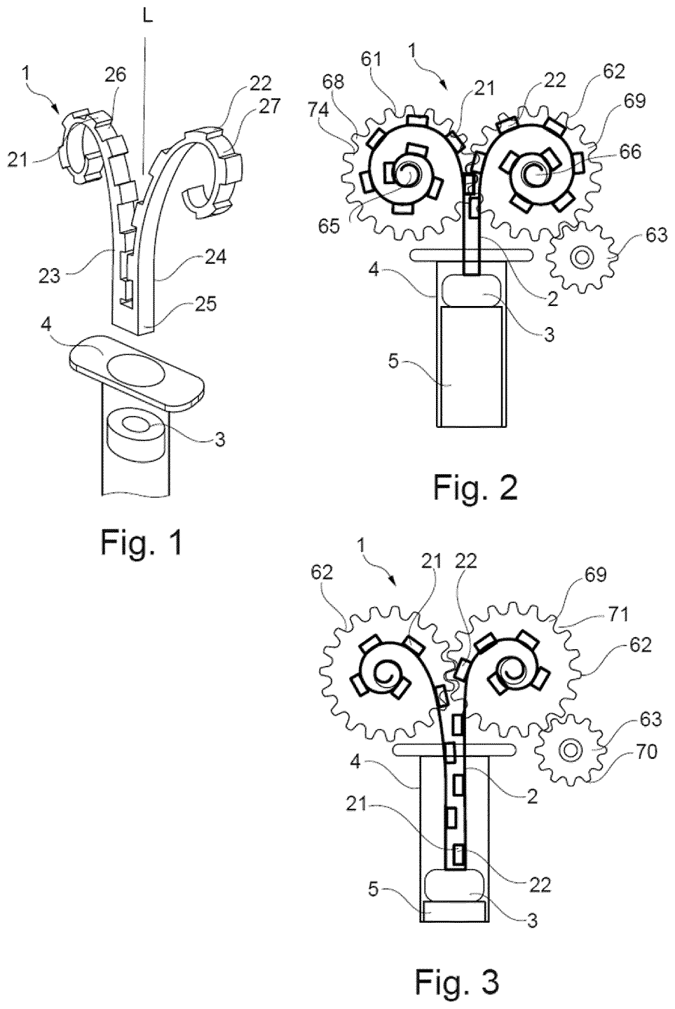

FIG. 1 shows a plunger rod 1 comprising a base 25 at the proximal end from which two arms 23, 24 extend distally. At the proximal end the two arms 23, 24 are joined together by the base 25. Or in other words, the two arms are joined together at the proximal end forming the base 25. In a first rolled-up configuration as shown in FIG. 1, a first arm 23 is rolled-up outwardly in a perpendicular direction with respect to a longitudinal axis of the medicament delivery device, the longitudinal axis is the axis L, and the axis L is a central axis of the plunger rod and syringe as shown in FIG. 1. In other words the first arm 23 rolls up in a direction away from the axis L. The second arm 24 of the plunger rod is also rolled-up outwardly, i.e. in a direction away from the longitudinal axis L. In other words, the first arm 23 is rolled-up in a counter clockwise direction and the second arm 24 is rolled up in a clockwise direction, when looked at from the side as in FIGS. 2 and 3. In this example the plunger rod is an integral part comprising the two arms and the base; in other words the two arms 23, 24 are fixedly joined together through the base 25.

In another example the two arms 23,24 further comprise a locking mechanism configured to removably or fixedly engage the two arms. The engagement with the locking mechanism of the two arms forms the base 25 of the plunger rod. For example, the first arm 23 may comprise a recess with locking elements, for example threads, a snap fit or any other suitable locking element for this purpose. The second arm 24 comprises a protrusion configured to engage with the recess of the first arm 23 to reversibly or irreversibly attach the two arms together, thereby forming a base.

In the example shown in FIG. 1, the first and second arms 23, 24 comprise an engagement mechanism in form of one or more protrusions 21 extending from the side of the first arm 23, that is facing the second arm 24 and one or more protrusions 22 extending outwardly from the side of the second arm 24, that is facing the first arm 23. Further, the first arm comprises one or more first spacings 26, wherein a spacing 26 separates adjacent protrusions 21 on the first arm 23 (i.e. the protrusions are spaced apart from one another). The second arm 24 comprises one or more second spacings 27, wherein a spacing 27 separates adjacent protrusions 22 on the second arm 24 (i.e. the protrusions are spaced apart from one another). The one or more protrusions 21 and the one or more first spacings 26 of the first arm 23 are arranged such that each of the one or more protrusions 21 of the first arm engages with a corresponding spacing 27 on the second arm, and each of the one or more spacings 26 of the first arm receives a corresponding protrusion 22 of the second arm 24. The length in the longitudinal direction of each of the first and second spacings 26,27 corresponds to the length of the respective corresponding protrusion each of the spacings is configured to receive. In this way the two arms 23,24 of the plunger rod engage with each other, and the plunger rod is formed by the two engaged arms. Thereby the plunger rod can extend in the longitudinal direction when the two arms are unrolled. The two arms engage with each other when a pair of a protrusion and a respective spacing engage with each other, as shown in FIG. 3. The plunger rod is extended by a length of the engaged protrusion in the proximal direction at the base 25 in this case. A subsequent extension in the longitudinal direction of the plunger rod is based on an engagement of a subsequent pair of a protrusion and a respective spacing.

As is shown in FIGS. 2 and 3, the transition from a first rolled-up configuration to a second extended configuration of the plunger rod 1 allows a longitudinal extension of the plunger rod. When the plunger rod 1 is arranged in a medicament delivery device, the base 25 of the plunger rod 1 is arranged distal (either adjacent to as shown in FIG. 2, or with a gap between the base and the plunger) to a plunger (stopper) 3, the plunger 3 being arranged in the medicament container, for example in a syringe comprising a barrel 4. The syringe comprising the barrel 4 in this example is a prefilled syringe that is received within a medicament delivery device. The prefilled syringe comprises a medical fluid 5. When the plunger rod 1 is transformed from the first up-rolled configuration, as shown in FIG. 2, to the second extended configuration, the plunger rod 1 displaces the plunger 3 in the proximal direction towards the medicament delivery member. The transformation will be described in further detail further below. The medical fluid 5 thus is displaced by the plunger under the action of the plunger rod towards the injection site. The medical fluid can be expelled by the medicament delivery member.

In this example shown in FIGS. 1 to 3 each of the first and second protrusions are the same length. An underlying effect is that the displacement volume of the medical fluid 5 which is displaced by the plunger rod 1 is proportional to the length of the protrusion 21 or 22 which engages each time with a respective spacing 26, 27. In this case, each time a protrusion engages with a spacing the plunger rod is extended longitudinally in the proximal direction by the length of the protrusion, which has engaged with the respective spacing (i.e. the distance from finishing the engagement of a protrusion with a spacing to starting the engagement of the next protrusion with the next spacing provides this length of extension in the proximal direction). When the length in the longitudinal direction of the protrusion is equal for all protrusions, the displaced volume of the medicament delivery device is each time the same, and the displacement volume is proportional to the length of a pair of engaged protrusion and corresponding spacing. This could be an advantage for a multi-dose medicament delivery device, as the medical fluid to be expelled on each dose can be metered with the plunger rod displacement.

In another example the length of a protrusion and its corresponding spacing may vary. For example, the length may decrease or increase or alternate. This could be an advantage for a multi-dose medicament delivery device with varying successive doses; for example, a medicament delivery device which expels different doses of the medical fluid successively. An example is a medicament dose that is taken at different dosages at different times. Instead of providing a single use medicament delivery device for each dose with the corresponding medicament fluid volume, or instead of a medicament delivery device with a dose setting mechanism that requires the actuation of the user for setting (i.e. choosing) the dose prior to injection, a multi-dose medicament delivery device could be provided with automatic dose setting for successive medicament deliveries to a user. This can provide a more sustainable solution and a more user-friendly solution.

As illustrated in FIG. 2, the distal end of the first arm 23 is attached to a first holding mechanism 65 and is rolled-up counter clockwise around the first holding mechanism 65. The distal end of the second arm 23 is attached to a second holding mechanism 66 and is rolled-up clockwise around the second holding mechanism 66. The first and second holding mechanisms 65, 66 may comprise a rod. For example, the rod is attached to a part of the medicament delivery device and the rod is rotatable such that the respective arm can be unrolled from the rod. Alternatively, the rod is not rotatable but is arranged inside guiding ribs such that when the rod moves along the guiding ribs the arms can be unrolled.

Further, the first holding mechanism 65 can be in the form of a first support wheel 61, for example a cog wheel, wherein a holding element, for example a rod or an axle is arranged in the centre of the first support wheel 61. The first support wheel 61 comprises circumferentially outwardly protruding first teeth 68 on the outer circumference of the first support wheel 61, wherein the first teeth 68 are separated by third spacings 71. In other words, the first support wheel is a gear wheel.

The second holding mechanism 66 can be in the form of a second support wheel 62, wherein a holding element, for example a rod or an axle, is arranged in the centre of the second support wheel 62. The second support wheel 62 comprises circumferentially outwardly protruding second teeth 69 on the outer circumference of the second support wheel 62, wherein the second teeth 69 are separated by fourth spacings 73. In other words, the second support wheel is a gear wheel.

Further, at least one of the support wheels 61,62 is engaged with an actuation gear wheel 63. The actuation gear wheel 63 comprises circumferentially outwardly protruding actuation teeth 70 on the outer circumference of the actuation gear wheel 63. The actuation teeth 70 are configured to engage with the third spacings 71 or the fourth spacings 73 of the first or second support wheels 61,62 respectively. When actuation teeth 70 engage the spacings 73 or 71, the actuation gear can be arranged next to the first support wheel or next to the second support wheel. Alternatively, two actuation gear wheels may be engaged with each of the support wheels. In the following the example as shown in FIG. 3 is described in more detail.

The actuation gear wheel 63 may be accessible to the user and can be actuated by the user; alternatively, the actuation gear wheel 63 is arranged with an automated actuation. An example of an automated actuation is a release button, which, when actuated, unlocks or releases a biasing mechanism such as a spring (e.g. a clock spring) or another mechanism that can rotate the actuation gear wheel. The biasing mechanism forces the actuation gear wheel to rotate.

When the actuation gear wheel 63 engages with spacings 71 of the second support wheel 62, and is turned clockwise then the support wheel to which the actuation gear wheel 63 is engaged, the support wheel is rotated counter clockwise and allows the second arm 24 to unroll, thereby the one of the protrusions 22 engages with a corresponding spacing 26 of the first arm 23. This engagement forces the first arm to unroll and thereby the first support wheel 61 is rotated as the first arm 23 unrolls.

In an example the first support wheel 61 is further engaged with the second support wheel 62, thereby when the second support wheel 62 is rotated by the actuation gear wheel 63 then the spacings 71 of the second support wheel 62 engage with the first teeth 68 of the first support wheel 61. This rotates the first support wheel 61. The rotation of the actuation gear wheel 63 is translated to the first support wheel 61, the first support wheel 61 is rotated clockwise thereby unrolling the first arm 23.

In another example the two plunger rod arms 23, 24 are biased when they are rolled up, and are held at the distal end in the rolled-up configuration. When the first and second holding mechanism releases the first and the second arm the arms snap back in to the unbiased extended state. In this example the holding mechanism can release the arms, which will unbias on their own, and therefore the support wheels and the actuation gear wheel can be omitted. This can provide a fast extension of the plunger rod which can be compactly rolled up.

All the above presented examples can be implemented in medical delivery devices such as on-body medical delivery devices, manual medical delivery devices and auto-injectors.

Various modifications to the embodiments described are possible and will occur to those skilled in the art without departing from the invention which is defined by the following claims.

Examples and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, any apparatus, element or method “configured to” perform a specified function is indeed capable of performing the specified function without any alteration, rather than merely having the potential to perform the specified function after further modification. In other words, the apparatus, element, or method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

Further, in the following description, the wording “medicament delivery device” will be used. In this context, medicament delivery devices may include a number of devices capable of delivering certain doses of medicament to a user. The medicament delivery devices may be either disposable or re-usable and may be provided with medicament containers suitably arranged for specific drugs in specific forms.

While various aspects and embodiments have been disclosed herein, other aspects and embodiments will be apparent to those skilled in the art. All embodiments within and between different aspects of the devices and methods can be combined unless the context clearly dictates otherwise. The various aspects and embodiments disclosed herein are for purposes of illustration and are not intended to be limiting, with the true scope being indicated by the claims.

1-13. (canceled)

14. A plunger rod for a medicament delivery device, the plunger rod comprising:
a base at a proximal end of the plunger rod;
a first arm extending from the base; and
a second arm extending from the base,
wherein each of the first arm and the second arm is flexible and is configured to transform from a first rolled-up configuration to a second extended configuration.

15. The plunger rod of claim 14, wherein each of the first arm and the second arm comprises an engagement mechanism.

16. The plunger rod of claim 16, wherein the engagement mechanism comprises two or more first protrusions on the first arm and two or more second protrusions on the second arm,
wherein each of the two or more first protrusions is spaced apart from the other first protrusions, and
wherein each of the two or more second protrusions is spaced apart from the other second protrusions.

17. The plunger rod of claim 16, wherein the two or more first protrusions are offset from the two or more second protrusions so that the two or more first protrusions fit between the two or more second protrusions when the first arm and the second arm are in the second extended configuration.

18. A plunger rod assembly comprising:
the plunger rod of claim 1;
a first support wheel configured to roll up the first arm; and
a second support wheel configured to roll up the second arm.

19. The plunger rod assembly of claim 18, wherein the first support wheel comprises a first holding mechanism for holding the distal end of the first arm, wherein the holding mechanism is configured to hold the first arm when the first arm is rolled up on the first support wheel, and wherein the second support wheel comprises a second holding mechanism for holding the distal end of the second arm, and the second holding mechanism is configured to hold the second arm when the second arm is rolled up on the second support wheel.

20. The plunger rod assembly of claim 19, wherein the first support wheel is a gear wheel comprising circumferentially outwardly protruding first teeth spaced out around the outer circumference of the first support wheel.

21. The plunger rod assembly of claim 20, wherein the second support wheel is a gear wheel comprising circumferentially outwardly protruding second teeth spaced out around the outer circumference of the second support wheel.

22. The plunger rod assembly of claim 18, further comprising:
an actuation gear wheel, wherein the actuation gear wheel comprises circumferentially outwardly protruding actuation teeth on the outer circumference of the actuation gear wheel and is configured to engage either the first support wheel or the second support wheel.

23. The plunger rod assembly of claim 22, wherein the actuation gear wheel is configured to turn the first support wheel or the second support wheel when actuated, thereby unwinding the second arm and moving the base, which results in the first arm also unwinding, thereby transforming both the first arm and the second arm from the first rolled-up configuration to the second extended configuration, wherein in the second configuration, the first arm and the second arm form together an extended rigid plunger rod.

24. The plunger rod assembly of claim 18, wherein the base is configured to exert force and displace a plunger within a barrel of a medicament container when the first arm and the second arm move from the first coiled up configuration to the second extended configuration, thereby expelling a medical fluid.

25. A medicament delivery device comprising:
the plunger rod of claim 1; or
the plunger rod assembly of claim 18.

26. The medicament delivery device of claim 25, comprising:
a medicament container;
a plunger in the medicament container;
a housing; and
a medicament delivery member.
